(19) United States
(12) Patent Application Publication
(10) Pub. No.: US 2010/0180890 A1
(43) Pub. Date: Jul. 22, 2010

(54) INHALER

(75) Inventor: Toshiyuki Nobutani, Yokohama-shi (JP)

Correspondence Address: FITZPATRICK CELLA HARPER & SCINTO, 1290 Avenue of the Americas, NEW YORK, NY, 10104-3800, US

(73) Assignee: Canon Kabushiki Kaisha, Tokyo (JP)

(21) Appl. No.: 12/438,265

(22) PCT Filed: Aug. 28, 2007
(86) PCT No.: PCT/JP2007/067099

§ 371 Date: Feb. 20, 2009

(30) Foreign Application Priority Data
Sep. 29, 2006 (JP) 2006-266652

(51) Int. Cl. A61M 15/00; H04N 7/18

(52) U.S. Cl. 128/203.12; 348/61; 348/E07.085

(57) ABSTRACT

An inhaler according to the present invention includes the following so that a user can visually confirm the ejection status of medicine: an air flow path communicating with a suction port through which a user inhales medicine; a medicine ejection portion ejecting the medicine supplied to the air flow path; an image sensor picking up the inside of the air flow path; a memory storing image data being the output of the image sensor; writing means storing the image data in the memory; and reading means reading the image data stored in the memory: wherein, the writing means is operated in synchronization with the timing of ejecting the medicine or after passing a predetermined time from ejecting the medicine.

### TECHNICAL FIELD

The present invention relates to an inhaler configured to be carried by a user and helping the user inhale medicine.

### BACKGROUND ART

An inhaler has been developed in which a minute medicine droplet is ejected into an air flow path through which air inhaled through a mouthpiece flows by using ejection principle of inkjet system (refer to International Publication WO1995/01137, International Publication WO2002/04043 and Japanese Patent Application Laid-Open No. 2004-97617). Such inhaler has an advantage of accurately spraying a predetermined amount of medicine with a uniformed particle size. Procedures have been realized for users utilizing information database such as an electronic medical recording system. An inhaler may be provided with storage means storing users personal information including information on users medical records and prescription, for example, and droplets of medicine can be ejected according to inhalation profiles, thereby users can inhale medicine according to information on prescription.

According to the above, there is no need of using medical appliances such as injection unlike before when medicine is doused out, so that the inhaler can be easily operated without expertise and the users are freed from enduring the pain of an injection needle.

On the other hand, an inhaler described in Japanese Patent Application Laid-Open No. 2004-97617 performs a preliminary ejection in which no inhalation is made before an inhaling medicine is ejected and detects the ejection of the medicine by detecting means to improve the reliability of the inhaler, thereby removing user's anxiety as to whether liquid droplets are properly ejected.

As a means of detecting the ejection of medicine, there have been known an optical method of detecting reflected light, refractive light, transmitted light, scattered light or laser in the atmosphere of ejected medicine. In addition to the above, there are known a method of detecting change in temperature in the atmosphere of ejected medicine using infrared rays and a method of using a humidity sensor detecting change in electrostatic capacity or impedance in the atmosphere of ejected medicine.

The above related art processes the output of various sensors by calculation means, determines the ejection status and displays the results on a display portion. The art, however, has a problem in that a user and medical service worker cannot directly observe the status where medicine is ejected.

### DISCLOSURE OF THE INVENTION

The present invention has been made in view of the problems with the related art and has for its purpose to provide an inhaler capable of imaging the ejection of medicine, thereby a user can directly observe the status where medicine is ejected.

To achieve the above purpose, an inhaler according to the present invention includes:

an air flow path communicating with a suction port through which a user inhales medicine;

a medicine ejection portion ejecting the medicine supplied to the air flow path;

an image sensor picking up the inside of the air flow path;

a memory storing image data being the output of the image sensor;

writing means storing the image data in the memory; and

reading means reading the image data stored in the memory:

wherein,

the writing means is operated in synchronization with the timing of ejecting the medicine or after passing a predetermined time from ejecting the medicine.

The present invention configured as the above produces the effects described below.

An user himself/herself or medical service worker can ascertain whether medicine is normally ejected at the time of inhaling through image data displayed on a display device. That is to say, an user himself/herself or medical service worker can view the image data displayed on the display to determine whether the situation is normal, further improving reliability and providing the user with a feeling of security.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic perspective view of an inhaler according to one embodiment of the present invention.

FIG. 2 is a schematic front view illustrating the state where a medicine cartridge and mouthpiece are removed from the main body of the inhaler illustrated in FIG. 1.

FIG. 3 is a perspective view of the medicine cartridge used in the inhaler of the present invention.

FIG. 4 is a block diagram of a control portion in the inhaler according to one embodiment of the present invention.

FIG. 5 is a diagram illustrating a remote control system using the inhaler of the present invention.

### BEST MODES FOR CARRYING OUT THE INVENTION

Preferred embodiments of the present invention will now be described in detail in accordance with the accompanying drawings.

The inhaler according to the present invention is characterized by being provided with an image sensor so that a user can directly confirm the ejected medicine. A configuration of the inhaler according to the present invention is described below.

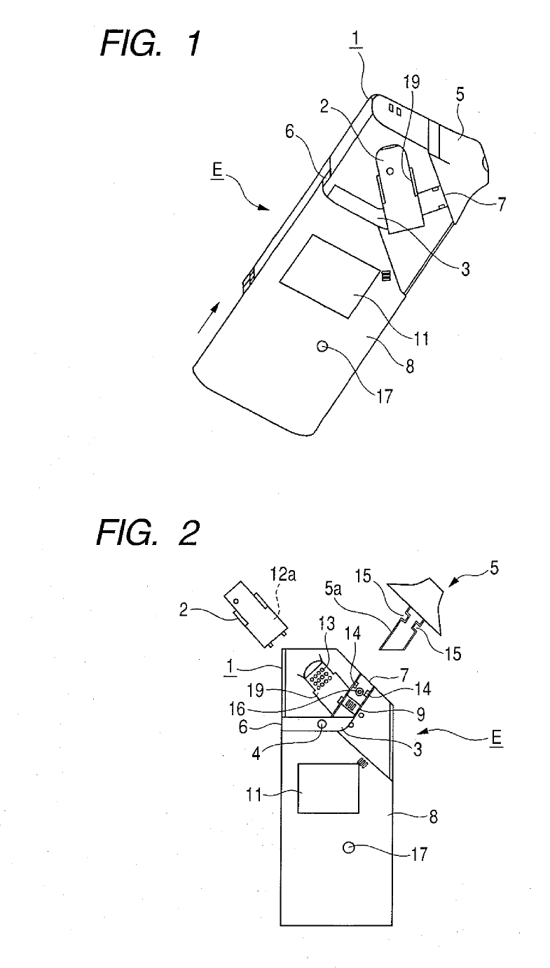

FIG. 1 is a schematic perspective view of an inhaler according to one embodiment of the present invention. FIG. 2 is a schematic front view illustrating the state where a medicine cartridge and mouthpiece are removed from the main body of the inhaler illustrated in FIG. 1.

As illustrated in FIGS. 1 and 2, an inhaler E of the present embodiment includes a main body 1, medicine cartridge 2 and mouthpiece 5 detachably provided on the main body 1 and cover 8 for covering the external face of the main body. Although the mouthpiece 5 as a suction port through which a user inhales medicine is presumed to be sucked by the mouth, the present invention is not limited this, a nosepiece for inhaling through the nose may be applied.

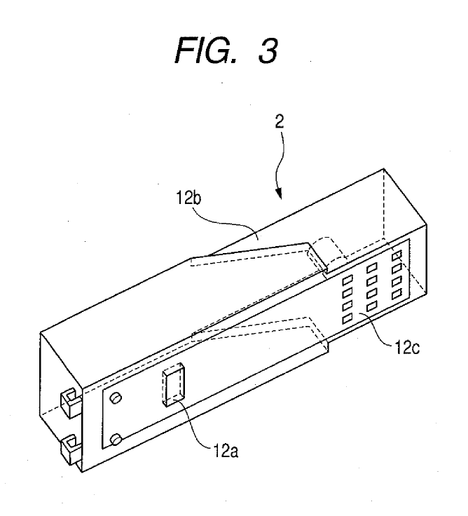

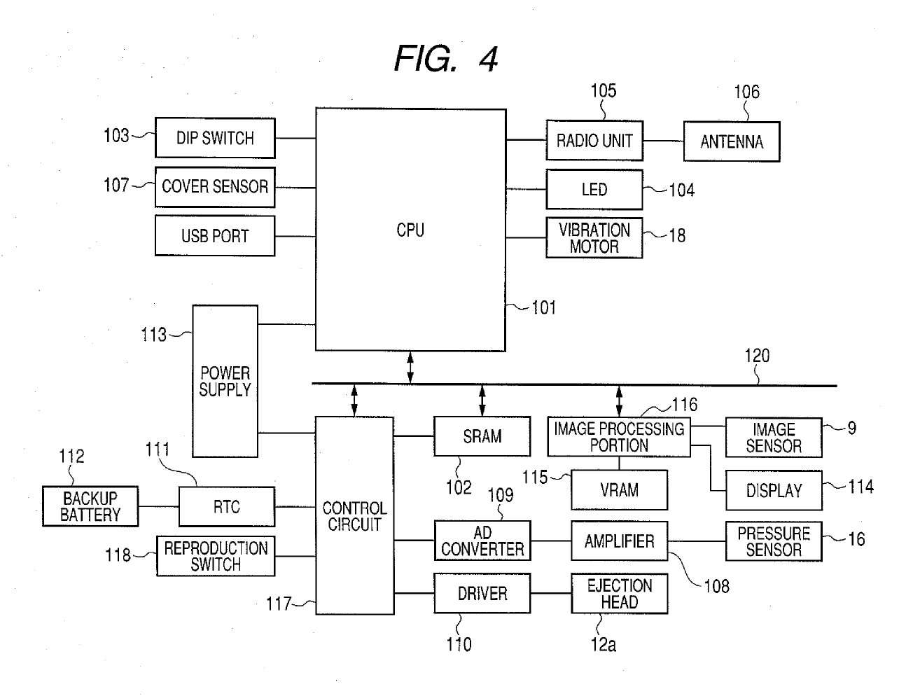

The medicine cartridge 2 is detachably fitted into a fitting portion 19 provided on the side of main body 1. FIG. 3 is a perspective view of the medicine cartridge 2. As illustrated in FIG. 3, the medicine cartridge 2 includes a reservoir 12b for containing medicine, ejection head 12a being a medicine ejection portion for ejecting medicine and electrical connection portion 12c for supplying a heater provided on the ejection head 12a with electric power for generating thermal energy or driving signal. Thus, the reservoir and the ejection head may be integrally formed into a cartridge, or otherwise, the reservoir and the ejection head may be separately formed.

The medicine cartridge 2 is fitted into the fitting portion 19 to connect the electrical connection portion 12c to an electric contact 13 of the fitting portion 19 to receive power and various electric control signals from the battery on one side of the main body 1 and a control circuit (not shown).

The main body 1 is provided with an air flow path 3 as a space for passing medicine ejected from the medicine cartridge 2 and guiding the medicine to the mouthpiece 5 being a suction port. In other words, the mouthpiece is adapted to be connected to the air flow path 3. When the medicine cartridge 2 is fitted into the fitting portion 19, the ejection head 12a is provided to expose inside the air flow path 3.

The air flow path 3 is desirably provided with light emitting means 4 to illuminate the medicine (misty liquid droplets at the time of ejecting medicine) ejected from the head portion 12a. The light emitting means 4 such as a high brightness LED for emitting light beams across the air flow path 3 in an appropriate direction is disposed in the vicinity of the head portion 12a for the case where the medicine cartridge 2 is fitted into the ejection-unit fitting portion 19. The mouthpiece 5 used by the user at the time of inhaling has a communicating portion 5a communicating with the air flow path 3. A pair of concaves 15 provided on both sides of the communicating portion 5a is detachably engaged with a pair of convexes 14 provided on opposing wall faces of the air flow path 3.

Furthermore, the cover 8 is slid in the direction indicated by an arrow from an illustrated position and closed to stop an opening portion on the upper face of the air flow path 3, thereby fixing the medicine cartridge 2 to the main body 1.

In the present embodiment, the air flow path 3 is bent on the way from an inlet 6 opening at one side of the main body 1 to an outlet 7 not to interfere with other constituents. If there are no other interfering constituents with which the air flow path 3 interferes, the air flow path 3 may be formed in a straight line from the inlet 6 to the outlet 7.

The mouthpiece 5 is attached to the outlet 7 of the air flow path 3 in the inhaler E, a power supply button 17 is pressed and then the user performs an inhaling operation. Then, medicine is ejected from the ejection head 12a of the medicine cartridge 2. Inhalation causes air to enter the air flow path 3 from the inlet 6 and medicine accompanying air flow generated in the air flow path 3 is ingested into the human body through the mouthpiece 5 attached to the outlet 7. A pressure sensor 16 is provided inside the air flow path 3 to detect the negative pressure generated inside the air flow path 3 by the inhalation of the user. That is to say, the pressure sensor enables detecting the inhalation operation of the user. When the inhalation operation of the user starts and a negative pressure (related to a inhalation rate and flow rate) detected by the pressure sensor 16 reaches a decision level at which medicine should be ejected, the ejection of medicine is started from the ejection head 12a by the control of a control portion.

A method of starting the ejection of medicine may depend on a switch which the user can instruct to start at user's discretion as well as on the pressure sensor 16 whereby inhalation is detected as described above. The light emitting means 4 may be adapted to light in synchronization with the start of ejection, or to light by the user by means of another switch.

The term “medicine” used in the present invention refers to not only medicine of pharmaceutical compound indicating pharmacological and physiological action, but also perfume, scenting and flavoring agent, dye and pigment in addition to pharmaceutical compound. The medicine may be liquid or powder.

The term “liquid medicine” used in the present invention refers to medicine in liquid form or liquid medium containing medicine. The liquid medicine may contain arbitrary additives. The state of medicine in liquid may be any of dissolution, dispersion, emulsification, suspension or slurry and the medicine may be desirably uniformed in liquid.

If liquid medicine is used as medicine, the principal medium in the liquid is desirably water or organic substance. Water is desirably the principal medium in consideration that a living body is dosed with liquid medicine.

In the present invention, the ejecting principle in a medicine ejecting portion (or the ejection head) is not limited to anything specific and the medicine ejecting portion includes an arbitrary ejecting energy generating element. An electrothermal transducer for providing medicine with thermal energy or an electromechanical transducer for providing medicine with mechanical energy are taken as examples of an ejecting energy generating element. In other words, the following are exemplified as a method of ejecting medicine: a method of ejecting medicine by providing medicine with thermal energy using the electrothermal transducer (thermal jet method); and a method of ejecting medicine using vibrational pressure of the electromechanical transducer (for example, piezo-electric device) providing medicine with mechanical energy. The ejection method may be selected according to the kind of medicine.

The use of the thermal jet method enables increasing accuracy and reproducibility of aperture of the ejection port, heat quantity of thermal pulse used for ejection, size of a micro heater as an electrothermal transducer in individual medicine cartridge. Thus, narrow droplet diameter distribution can be achieved. The ejection head is low in manufacturing cost, so that the thermal jet method is highly applicable to a small apparatus of which ejection head needs to be frequently replaced. Consequently, if a liquid ejection apparatus is required to have a high portability and convenience, an ejecting apparatus of the thermal jet type is desirably used.

The following describes the case where liquid medicine is ejected and turned into misty liquid droplet. An image sensor 9 is arranged at a position where the sensor picks up at least part of the air flow path 3 from the vicinity of the ejection head 12a to the outlet 7. A display portion 11 as a display device is disposed on the side of the cover 8 and is configured by a dot matrix display liquid crystal display, for example. While liquid droplets are ejected along with the inhalation operation, the image sensor 9 picks up ejected liquid droplets and the image data being the output of the image sensor 9 is stored in a memory (not shown) arranged in the main body 1. Contents of the image data stored in the memory are displayed on the display portion 11 by operating a reproduction switch (not shown). Displayed contents may be a moving or a still image through which the normally ejected liquid droplets can be recognized. In addition, displayed contents may be an image processed to make liquid droplets more visible. The term “process” refers to “image processing” to emphasize liquid droplets. Operating the reproduction switch allows the same images to be repeated two or more times.

A CCD image sensor and CMOS image sensor may be exemplified as the image sensor 9.

The image sensor 9 may be disposed anywhere in the air flow path 3 as long as ejected liquid droplets in the present embodiment can be picked up. Light emitted by the light emitting means 4 does not always need to be visible rays as long as the image sensor 9 can pick up the light and it can be displayed on display means.

According to the present embodiment, the user can ascertain the ejecting status of liquid droplets at the time of inhalation through images displayed on the display portion 11. Thus, the fact that the user can ascertain the actual ejecting status of liquid droplets provides him/her with a feeling of security.

FIG. 4 is a block diagram of a control portion in the inhaler according to the present invention. A

CPU 101 being a calculation process unit includes a flash ROM for storing programs. A SRAM 102 is a readable/writable memory to temporally store data when the programs are operated and is connected to the CPU 101 through a bus 120. A DIP switch 103 is connected to the port of the CPU 101, sets ON/OFF information and causes the CPU 101 to capture the

ON/OFF information. An LED 104 is a display device for informing a user or maintenance operator of the status of the apparatus. A radio unit 105 serves to manage transfer of the status of the apparatus, stored contents and data to a host such as a personal computer or radio communication for receiving data from the host. An antenna 106 is connected to the radio unit. A cover sensor 107 detects the opening and closing of the cover 8. An amplifier 108 performs the level conversion and amplification of output of the pressure sensor 16. An AD converter 109 converts the analog output of the amplifier 108 to a digital signal. A driver 110 controls the ejection head 12a. An RTC 111 has functions of calendar and watch. A battery 112 serves as a backup battery for the RTC 111. A power supply 113 generates various voltages supplied to electric circuits and includes a main battery, charging circuit, reset circuit and power supply switch. A reproduction switch 118 causes the display device to display picked up image data thereon.

A control circuit 117 processes the output signal or the input signal to various blocks and is connected to the CPU 101 through the bus 120.

Reference numeral 114 denotes a display. A VRAM 115 serves as a memory for storing image data. An image processing portion 116 is equipped with writing means and reading means for the VRAM 115. The image processing portion 116 has functions to capture the image data being the output of the image sensor 9, process the image data if required, control writing the image data or processed image data into the VRAM 115 and control reading the contents from the VRAM. In addition to the above, the image processing portion 116 has integrated functions to process read image data if needed, control displaying the read image data or processed image data on the display 114 and communicate the image data with the CPU.

Operating the power supply button 17 causes the power supply 113 to output a reset signal to the CPU 101 to initialize the CPU 101. Then, the program stored in the flash ROM inside the CPU starts operation, the status of the DIP switch 103 is captured and operation is started as a normal operation mode if a special setting is not set.

The inhalation of the user varies the output of the pressure sensor 16, i.e., the pressure inside the air flow path 3, which is transferred to the CPU 101 through the amplifier 108, AD converter 109 and the control circuit 117. If variation in pressure exceeds a predetermined threshold, the CPU applies a voltage to a vibration motor 18 to vibrate the motor and sends a pulse signal to a head portion 12 through the control circuit 117 and driver 110, thereby ejecting liquid droplets contained in the medicine cartridge 2. In addition, the CPU 101 sends a command to the image processing portion 116 through the bus 120 and the image processing portion 116 captures the image data being the output of the image sensor 9 by the command and stores the image data in the VRAM 115. The control of writing the image data in the VRAM 115 is desirably synchronized with the timing of ejection. As a method of synchronizing the control of writing the image data with the timing of ejection, the timing of starting the ejection of liquid droplets may be synchronized with that of capturing the image data, alternatively, a certain time difference may be provided therebetween. For example, the capture of the image data may be started 50 milliseconds before ejection is started to compare the image data before and after ejection is started, or otherwise, the capture of the image data may be started 300 milliseconds after ejection when the mist is sufficiently stabilized has been started. In that case, the capacity of memory to be captured can be reduced. If the ejection head 12a is away from the image sensor 9 by a certain distance, the capture of the image data is desirably started with a certain time difference provided. Since there is a time difference between the start of ejection at the ejection head 12a and the arrival of mist at the pick-up area of the image sensor 9, the capture of the image data is started with the time difference provided, thereby not to wastefully consuming a memory capacity. The time difference depends on flow rate of air generated inside the air flow path, that is, on strength with which the user inhales, so that the time difference may be set for each user.

The time difference between the timing of start of ejection and the timing of capture of the image data can be realized by preparing two thresholds of output of the AD converter 109, for example.

The image data is captured from the image sensor 9 and then calculated if required before stored in the VRAM. The term “calculation” herein refers to the compression of the image data or emphasis of part of liquid droplets in the image to make them more visible. In order to emphasize part of liquid droplets in the image to make them more visible, there exists a method in which a variation is detected for each frame, for example, and the variation part is overwritten (integrated) and calculated. The emphasizing method is broadly interpreted herein, and the variation part may be highlighted, color may be added thereto or a border portion may be indicated by frames.

Operating the reproduction switch 118 sends the information to the CPU 101 through the control circuit 117. The CPU 101 sends a command to the image processing portion 116 based on the information. The image processing portion 116 receives the command and reads the image data stored in the VRAM 115 and causes the display 114 to display the image data.

Operating again the reproduction switch causes the CPU 101 to send a command again to the image processing portion 116. The image processing portion 116 receives the command and reads the image data stored in the VRAM 115 and causes the display 114 to display the image data. Thus, the image data can be repetitively displayed according to user's request.

The image data stored in the VRAM 115 is read and then calculated if required before displaying it on the display 114. As one example of calculation, there exists decoding of compressed data.

Simply operating the reproduction switch after inhalation enables the user to confirm the status of ejection at the time of inhalation. The user confirming the actual ejection of liquid droplets through images provides him/her with a feeling of security. Even if the user fails to view displayed images at the time of reproduction, operating again the reproduction switch enables him/her to repetitively view the images, which liberates him/her from tension at the time of reproduction.

In the present invention, the reading means does not need to be simultaneously operated with the writing means because the ejection of medicine at the time of an actual inhalation is picked up instead of a preliminary ejection performed prior to inhalation. That is to say, once the user captures the image data and stores it in the VRAM 115, he/she can read the image data any time after inhaled.

A second embodiment according to the present invention is described below.

In FIG. 4, the CPU 101 accesses data in the VRAM 115 through the image processing portion to analyze the data to calculate the existence of liquid droplets or the amount thereof ejected from the ejection head 12a at the time of inhalation. The calculation of amount of liquid droplets herein refers to not only discrimination whether the amount of liquid droplets is close to a prescribed amount or liquid droplets hardly exist, but also measurement for determining the percentage of liquid droplets relative to the prescribed amount. Incidentally, the prescribed amount means the amount of medicine to be ingested by the user, typically determined by a doctor's prescription and set in the inhaler in advance.

As one example of a calculation method, there exists a method in which, for example, a plurality of image data including different amounts of ejection are stored in advance in the flash ROM incorporated in the CPU 101 and compared to the image data in the VRAM 115 to determine the amount of ejection. The calculated results are stored in the VRAM 115 through the image processing portion 116. In this case, the calculated results are stored to be associated with the original image data used as a ground for the calculated results. A plurality of the associated pairs can be stored in the VRAM 115. The operation of the reproduction switch 118 allows displaying pairs selected from among a plurality of the pairs on the display 114. In this case, it is possible to display on the display 114 the selected image data at the time of ejection and information measured at that point as to whether the amount of liquid droplets is close to a normal amount or not. The user can read the image data as well as data related to the amount of ejection determined by the apparatus through the display 114. The pairs may be stored, for example, in the flash ROM incorporated in the CPU 101, instead of the VRAM 115. A non-volatile memory may be used as the VRAM 115. Thereby, the user can access and view the past data.

The measurement may be performed based on degree to which liquid droplets intercept an optical axis or sound wave as well as on so-called image data such as data of the image sensor 9 or the VRAM 115. In addition, decrease in medicine contained in the medicine cartridge may be measured to determine the amount of ejection.

Notifying the user of the image data at the time of ejection and measurement results on the existence of liquid droplets provides him/her with results determined by himself/herself as well as the apparatus. This provides the user with a feeling of security and contributes to improvement in reliability of determination result.

The embodiment aside from the above is the same as the aforementioned embodiment, so that description thereof is omitted.

A third embodiment of an inhaler according to the present invention is described below. Parts which are the same as the first and second embodiments are omitted from description and different parts are described.

In FIG. 4, the CPU 101 accesses data in the VRAM 115 through the image processing portion. Then, the CPU analyzes the data to determine as to whether ejection and inhalation is appropriate. For example, the CPU determines based on the image data capturing the ejection of liquid droplets from the ejection head 12a at the time of inhalation or liquid droplets in the flow path 3 as to whether the amount of ejected liquid droplets, a position where liquid droplets pass in the air flow path and a rate at which liquid droplets move in the air flow path are appropriate.

The amount of ejected liquid droplets can be measured by the same method as that previously described in the second embodiment. As a result, if the amount of ejection is less than the prescribed amount, it is determined to be “No.” If the amount of ejection is more than the prescribed amount, it is determined to be “Yes.” A predetermined threshold value may be specified for each user.

A position where liquid droplets pass in the air flow path is determined by comparing appropriate and inappropriate areas stored in advance in the flash ROM (not shown) incorporated in the CPU 101 with the image data in the VRAM 115. As a result, if an area where liquid droplets pass reaches the inappropriate area, it is determined to be “No”. If no, it is determined to be “Yes”.

If it is determined whether ejection is appropriate based on a rate at which liquid droplets move in the air flow path, the image data needs to be moving image. In this case, it may be determined as to whether a predetermined inhalation falls within appropriate rate.

If out of a plurality of the above-exemplified factors, plural ones are used, and if at least one factor is determined to be “No”, the whole of ejection may be determined to be “No”. Alternatively, it is determined for each of items.

The determined result is stored in the VRAM 115 via the image processing portion 116. The determined result in this case is associated with the original image data used as a ground for the determined results. A plurality of the associated pairs can be stored in the VRAM 115. The operation of the reproduction switch 118 allows displaying pairs selected from among a plurality of the pairs on the display 114. In this case, it is possible to display on the display 114 the selected image data at the time of ejection and information measured at that point as to whether the amount of liquid droplets is close to a normal amount or not. The user can read the image data as well as data related to appropriateness of ejection determined by the apparatus through the display 114. The pairs may be stored, for example, in the flash ROM incorporated in the CPU 101, instead of the VRAM 115. A non-volatile memory may be used as the VRAM 115. Thereby, the user can access and view the past data.

The measurement may be performed based on attenuation caused by liquid droplets intercepting an optical axis or sound wave or by Doppler effect as well as on so-called image data such as data of the image sensor 9 and the VRAM 115.

Notifying the user of the image data at the time of ejection and decision results by the apparatus provides him/her with results determined by himself/herself as well as the apparatus. This provides the user with a feeling of security and contributes to improvement in reliability of determination result.

A fourth embodiment of an inhaler according to the present invention is described below. Parts which are the same as the first to third embodiments are omitted from description and different parts are described.

A user starts inhaling to change the output of the pressure sensor 16. The change is transferred to the CPU 101 through the amplifier 108, AD converter 109 and control circuit 117. If the amount of inhalation exceeds a predetermined threshold value, the CPU applies a voltage to the vibration motor 18 to vibrate the motor. At the same time, the CPU sends a pulse signal to the ejection head 12a through the control circuit 117 and driver 110 to cause the ejection head to eject medicine contained in the medicine cartridge 2. The CPU 101 also sends a command to the image processing portion 116 through the bus 120. The command causes the image processing portion 116 to capture image data from the image sensor 9 and store the image data in the VRAM 115, and the image data is transmitted by radio from the antenna 106 through the radio unit 105.

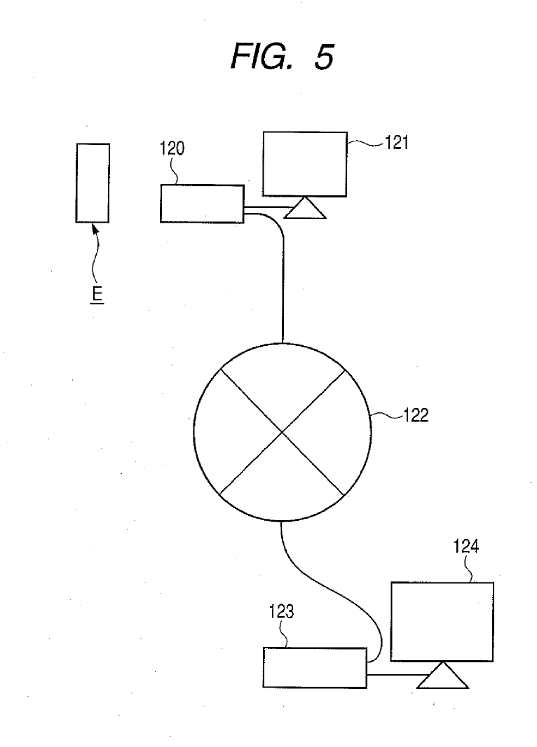

In FIG. 5, the personal computer 120 includes a radio communication function. Reference numeral 121 denotes a display device for the personal computer 120; 122, Internet network; 123, a personal computer provided in a medical institution; and 124, a display device for the personal computer 123. The personal computers 120 and 123 are remotely positioned from each other, but can be connected to the Internet network 122 to exchange data with each other. The image data transmitted by radio from the inhaler E is received by the personal computer 120 provided in the neighborhood and displayed on the display device 121. This enables the user to observe the status of liquid droplets ejected from the inhaler E while inhaling. The image data is sent to the personal computer 123 connected to the personal computer 120 through the Internet network 122. The personal computer 123 is capable of storing the image data in storage device such as a memory or hard disk and displaying the image data on the display device 124.

Thus, displaying the image data on the ejection of liquid droplets at the time of inhalation on the other display device 121 remotely located from the inhaler E equipped with the display portion 11 permits the user to simultaneously watch the image data of liquid droplets at the time of inhalation. That is to say, since the user can ascertain the ejection status in real time, he/she is provided with a feeling of security. In general, the screen of the display device 120 is far larger than that of the display portion 11, so that even people with impaired vision to some extent can ascertain the ejection status. If there is no need of simultaneously viewing the image data of liquid droplets at the time of inhalation, it is enabled to view a reproduced image after inhalation has been finished. Sending the image data to a remote place allows medical service worker to monitor the ejection status. This provides the user with a feeling of additional security because medical service worker monitors. In the present embodiment, the inhaler does not always need to be equipped with the display portion 11.

The present invention is applicable to various applications other than those for inhaling medicine. For example, various applications include an inhaler for a fragrance and article of taste such as nicotine and others requiring sure and hygienic ejection of liquid droplets.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2006-266652, filed Sep. 29, 2006, which is hereby incorporated by reference herein in its entirety.

1. An inhaler comprising:
an air flow path communicating with a suction port through which a user inhales medicine;
a medicine ejection portion ejecting the medicine supplied to the air flow path;
an image sensor picking up the inside of the air flow path;
a memory storing image data being the output of the image sensor;
writing means storing the image data in the memory; and
reading means reading the image data stored in the memory:
wherein,
the writing means is operated in synchronization with the timing of ejecting the medicine or after passing a predetermined time from the timing of ejecting the medicine.

2. The inhaler according to claim 1, further comprising a display device displaying the image data read by the reading means.

3. The inhaler according to claim 1, further comprising calculation means calculating the existence or amount of medicine ejected from the medicine ejection portion.

4. The inhaler according to claim 1, further comprising determining means determining based on the image data whether ejection or inhalation is appropriate.

5. The inhaler according to claim 3, wherein the image data is associated with results calculated by the calculation means based on the image data and stored in the memory.

6. The inhaler according to claim 4, wherein the image data is associated with results determined by the determining means based on the image data and stored in the memory.

7. The inhaler according to claim 1, wherein change in pressure in the air flow path is detected to generate the timing of ejecting the medicine.

8. The inhaler according to claim 1, wherein the reading means is not simultaneously operated with the writing means.
